(19) United States
(12) Patent Application Publication
(10) Pub. No.: US 2020/0056212 A1
(43) Pub. Date: Feb. 20, 2020

(54) FERMENTATION PROCESS FOR PRODUCING D-LACTIC ACID OR ITS SALTS

(71) Applicant: PTT Global Chemical Public Company Limited, Bangkok (TH)

(72) Inventors: Phatthanon PRASITCHOKE, Bangkok (TH); Kraileark KITTISURIYANONT, Bangkok (TH); Narong KAEWSUWAN, Bangkok (TH); Natthawut POOMSILA, Bangkok (TH); Nuttha THONGCHUL, Nonthalburi (TH)

(73) Assignee: PTT Global Chemical Public Company Limited, Bangkok (TH)

(21) Appl. No.: 16/345,991

(22) PCT Filed: Oct. 31, 2017
(86) PCT No.: PCT/TH2017/000077

§ 371 Date: Apr. 29, 2019

(30) Foreign Application Priority Data
Nov. 1, 2016 (TH) 1601006587

(51) Int. Cl. C12P 7/56

(52) U.S. Cl. CPC C12P 7/56

(57) ABSTRACT

This invention relates to a fermentation process for producing D-lactic acid or its salts. The said process comprises the following steps: cultivating of Sporolactobacillus laevolacticus bacteria strain by fermentation with sugarcane juice in order to obtain the seed culture; fermenting of the obtained seed culture in sugarcane juice, wherein the cultivation step is operated for the time that the final concentration of Sporolactobacillus laevolacticus strain in the cultivation step is in a range of 400 to 1,600 mg of dry cell per liter. The said process according to the invention can provide high productivity and yield of the D-lactic acid with a high optical purity. Moreover, the said process can be easily done and reduces the complicate steps.

### TECHNICAL FIELD

Biotechnology relates to a fermentation process for producing D-lactic acid or its salts using Sporolactobacillus laevolacticus bacteria strain.

### BACKGROUND OF THE INVENTION

It is well known that lactic acid has been used widely in plastic industry, food and drug industry, and cosmetic industry. For the plastic industry, lactic acid is widely used especially in the production of polyester, such as polylactic acid, or poly(lactic-co-glycolic acid). The polymers produced from lactic acid have advantages in their biodegradable and biocompatible. The said polymers can be used in many applications such as textile fiber, film, packaging, catgut, and scaffold in medical field.

At present, there are several production processes of lactic acid such as chemical synthesis and biotechnological method, which has several advantages including the use of natural resource as raw materials in microbial fermentation such as cassava corn, wheat, or sugarcane. The said raw materials can be reproduced continuously. Moreover, the microbial fermentation has one more advantage that can produce lactic acid with high optical purity.

Most of the productions on lactic acid in industrial scales are the productions by fermentation of sugar such as glucose, sucrose, maltose, or other carbohydrates such as starch or cellulose, wherein microbial capable for producing lactic acid are bacteria and fungi. At present, the L-lactic acid is widely produced in industrial scale.

However, The production of D-lactic acid in industrial scale does not disclosed. There are several problems such as most of naturally found bacteria or fungi producing L-lactic acid with high optical purity but very few can produce D-lactic acid. From this reason, there were only few studies for details of these bacteria or fungi. Therefore, the production development for D-lactic acid by naturally found microbial goes more difficultly.

Another problem in the production of polymer at present is that the raw materials are expensive. There is need for the production development of lactic acid in order to be efficient, reducing production cost, providing high yield and productivity. There had been attempts to develop microbial and microbial cultivation step that can be grown and reproduced well. Lactobacillus, Leuconostoc, and Streptococcus are well known bacteria strains in the production of lactic acid from sugar under an anaerobic condition which saves cost and provides product with higher concentration than fungi. Nevertheless, the said bacterial group is fastidious bacteria. Therefore, there is need for the use of several vitamins and essential amino acids in their growth. Moreover, the said bacterial group cannot produce enzymes for starch hydrolysis. Therefore, starch used as raw materials needed to be hydrolyzed into sugar before being used in fermentation. This causes higher production cost.

One attempt to reduce production cost of the lactic acid is to reduce a sugar purifying step by applying sugarcane juice in fermentation in order to produce the lactic acid. This can reduce a drying step and can utilize sugarcane juice to be fermentation liquid directly and can utilize nutrition in sugarcane juice as nutritional sources for microbial.

However, the achievement of using sugarcane juice to produce lactic acid especially the D-lactic acid is difficult because the microbial fermentation step is complicate. Therefore, the D-lactic acid product with a high concentration and productivity cannot be obtained.

Prachamorn et al. (The production of the lactic acid using sugarcane juice as precursor, KKU Research Journal, 2008, 8(3), July-September, 2008) disclosed the method of using sugarcane juice to produce the L-lactic acid by Lactobacillus genus bacteria. The results showed that the L-lactic acid at concentration higher than 23 g/L could not be produced although using sugarcane juice with sucrose concentration at 150 g/L fortified with a nitrogen compound from a yeast extract.

US2010/0112652 disclosed the fermentation step of the D-lactic acid in sugarcane juice using Sporolactobacillus genus bacteria. From the disclosure, it was found that the maximum concentration of the D-lactic acid was only 20 g/L although the nitrogen compound from the yeast extract had been added.

Kanwar et al. (Lactic acid production from molasses by Sporolactobacillus cellulosolvens. Acta Microbiologica et Immunologica Hungarica. 1995. 42 (4), 331-318) reported the result of fermenting molasses with Sporolactobacillus cellulosolvens in the production of the D-lactic acid providing final concentration of the lactic acid of 24.2 g/L which was not enough for the production in industrial scale.

Sawai et al. (Membrane-integrated fermentation system for improving the optical purity of the D-lactic acid produced during continuous fermentation. Bioscience and Biotechnology Biochemistry. 2011. 75 (12), 2326-2332) reported the fermentations of Sporolactobacillus inulinus, Sporolactobacillus laevolacticus, and Sporolactobacillus terrae with pure sucrose. Although, this could produce the high concentration of D-lactic acid, but it took 120 hours in fermentation caused the said process very high production cost.

From all reasons above, this invention aims to overcome said above problems by developing the fermentation process for producing the D-lactic acid using cheap raw material such as sugarcane juice. These developed process can provide high productivity and yield of D-lactic acid with high optical purity. Moreover, the said process can be easily done and reduces complicate steps

### SUMMARY OF INVENTION

This invention relates to the fermentation process for producing the D-lactic acid or its salts. The said process comprises the following steps: cultivating of Sporolactobacillus laevolacticus bacteria strain by fermentation with sugarcane juice in order to obtain the seed culture; fermenting of the obtained seed culture in sugarcane juice, wherein the cultivation step is operated for the time that the final concentration of Sporolactobacillus laevolacticus strain in the cultivation step is in a range of 400 to 1,600 mg of dry cell per liter. The said process according to the invention can provide high productivity and yield of the D-lactic acid with a high optical purity. Moreover, the said process can be easily done and reduces the complicate steps.

### BRIEF DESCRIPTION OF THE DRAWINGS

FIG. 1 shows nucleotide sequence of 16S rRNA gene of Sporolactobacillus laevolacticus bacteria strain, microbial accession number NITE ABP-02334.

FIG. 2 shows configuration of Sporolactobacillus laevolacticus bacteria strain, microbial accession number NITE ABP-02334.

FIG. 3 shows positive catalase test of Sporolactobacillus laevolacticus bacteria strain, microbial accession number NITE ABP-02334.

### DESCRIPTION OF THE INVENTION

### Definitions

Technical terms or scientific terms used herein have definitions as understood by an ordinary person skilled in the art unless stated otherwise.

Any tools, equipment, methods, or chemicals mentioned herein mean tools, equipment, methods, or chemicals commonly operated or use by the ordinary person skilled in the art unless explicated otherwise that they are tools, equipment, methods, or chemicals specifically in this invention.

Use of singular noun or singular pronoun with “comprising” in claims or specification refers to “one” and also “one or more”, “at least one”, and “one or more than one”.

Throughout this application, term “about” is used to indicate that any value presented or showed herein may potentially varied or deviated from individual operator implementing equipment or method including variation or deviation caused from changes in physical properties.

“Sugarcane juice” refers to a yield obtained from a sugarcane pressing process either with or without other processing. The said yield may comprises but not limited to sugar, starch, organic acid, polyphenol, protein, amino acid, fiber, chlorophyll, sulfated ash, gum, wax, SiO₂ form of silica, P₂O₅ form of phosphate, CaO form of calcium, K₂O form of potassium, MgO form of magnesium, or mixture thereof.

Sugar may be a monomolecular sugar selected from glucose, fructose, galactose, or mixture thereof; a dimolecular sugar selected from sucrose, lactose, maltose, cellobiose, or mixture thereof; a trisaccharide selected from raffinose, isomaltotriose, maltotriose, nigerotriose, kestose, or mixture thereof.

Throughout this invention, sugarcane juice refers to the solution obtained from sugarcane juice including a product obtained from subjecting sugarcane juice to the concentration process. An example of the solution obtained from sugarcane juice is the solution from evaporation process until obtaining a high concentration called a high-test molasses or a product obtained from precipitation of sugar in sugarcane juice and then redissolved the precipitate in water again.

“Microaerobic condition” refers to a condition that the amount of air is controlled to be limited without adding any further gas no matter the adding of air or adding of inert gas in order to replace the existing air.

Hereafter, the invention embodiments are shown without any purpose to limit any scope of the invention.

This invention relates to the fermentation process for producing the D-lactic acid or its salts, comprising the following steps:

(a) cultivating of Sporolactobacillus laevolacticus bacteria strain, microbial accession number NITE ABP-02334 in sugarcane juice in order to obtain the seed culture; and

(b) fermenting of the seed culture obtained from step (a) in sugarcane juice;

characterized in that step (a) is operated for the time that the final concentration of Sporolactobacillus laevolacticus strain in step (a) is in a range of about 400 to 1,600 mg of dry cell per liter, preferably in range of about 1,000 to 1,400 mg of dry cell per liter.

In one embodiment of the invention, step (a) is operated under an aerobic condition.

In one embodiment of the invention, step (a) has the starting concentration of Sporolactobacillus laevolacticus strain in a range of about 40 to 240 mg of dry cell per liter, preferably in the range of about 120 to 160 mg of dry cell per liter.

In one embodiment of the invention, step (b) is operated for the time in a range of about 12 to 50 hours, preferably in the range of about 45 to 50 hours.

In one embodiment of the invention, sugarcane juice in step (b) comprises a sugar that make the sugar concentration in step (b) in a range of about 4 to 15% by volume, wherein sugarcane juice may be the sugarcane juice obtained from processed or non-processed sugarcane juice.

In one embodiment, step (a) and (b) are operated at the temperature in a range of about 35 to 40° C., preferably operated at the temperature about 37° C.

In one embodiment, step (a) and (b) may further comprise the shaker mixing, wherein the shaker mixing speed in step (a) may be in a range of 10 to 1,200 rpm and the shaker mixing speed in step (b) may be in a range of 10 to 1,200 rpm.

In preferred embodiment, the D-lactic acid or its salts obtained from the fermentation process according to the invention have the optical purity more than 95%, more preferably the optical purity more than 99%.

Suitable Microbial According to the Invention

The suitable microbial according to this invention is Sporolactobacillus laevolacticus bacteria strain, especially the Sporolactobacillus laevolacticus bacteria strain deposited in NITE Patent Microorganisms Depositary (NPMD), Japan, accession number NITE ABP-02334.

In one embodiment, the Sporolactobacillus laevolacticus according to this invention can be grown under an aerobic condition at the temperature more than 30° C. and can produce the D-lactic acid or its salts with the high optical purity.

Preferably, the said Sporolactobacillus laevolacticus can be grown under an aerobic condition at the temperature in the range of 30 to 42° C., and can produce the D-lactic acid or its salts with the optical purity over 95%.

Most preferably, the said Sporolactobacillus laevolacticus can be grown under an aerobic condition at the temperature about 37° C., and can produce the D-lactic acid or its salts with the optical purity over 99%.

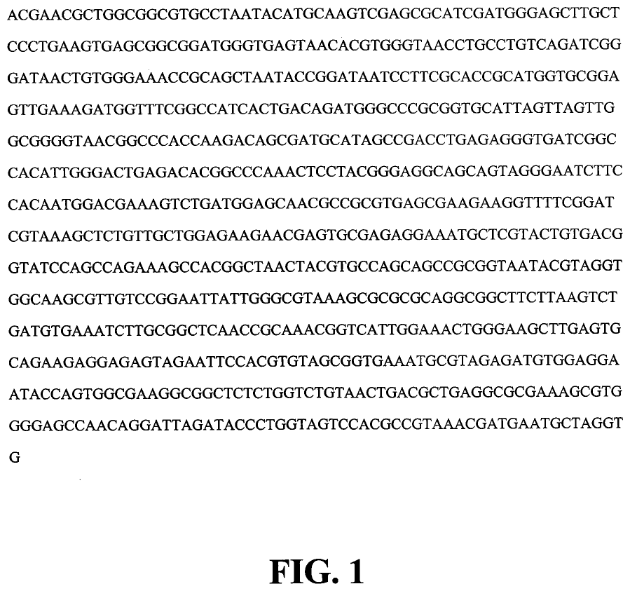

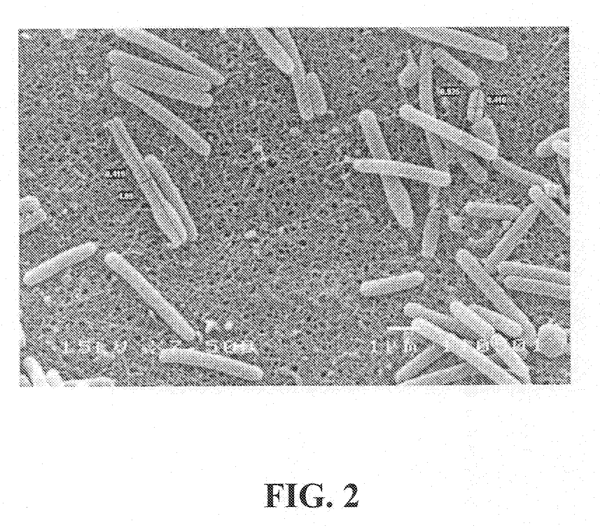

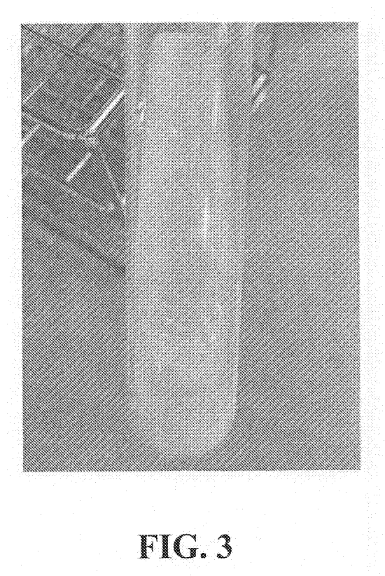

The Sporolactobacillus laevolacticus according to this invention is a gram-positive bacteria having nucleotide sequence of 16S rRNA gene as shown in FIG. 1, the configuration as shown in FIG. 2, and the positive in catalase test as shown in FIG. 3.

The Cultivation of Sporolactobacillus laevolacticus Bacteria Strain and the Fermentation of the Seed Culture Prepared from Sugarcane Juice to Produce the D-Lactic Acid

The following are examples of the cultivation of Sporolactobacillus laevolacticus bacteria strain according to this invention and the fermentation of the seed culture prepared from sugarcane juice for the production of the D-lactic acid. These examples are for a demonstrating purpose of this invention only, not intended to limit of this invention in any way.

The determination of each properties according to the invention was performed by methods and instruments as explained below, wherein each testing methods and instruments are methods and instruments commonly used and not intended to limit the scope of the invention.

The amount of sugar, lactic acid, and by-product were determined using high performance liquid chromatography (Agilent Technology) equipped with Biorad column, Aminex HPX-87H ion exclusion organic acid 300 mm×7.8 mm at the temperature of 50° C. The detector used was reflective index detector (Agilent Technology) as compared signal instrument to the standard solutions.

The optical purity was determined using chiral column (Sumipack, Sumichiral OA5000) at the temperature of 40° C. Copper sulfate (CuSO₄) was used as eluent. The flow rate was 1 mL/min. Signals were detected by UV Detector at the wavelength of 254 nm.

The optical density (OD) during cultivating or fermenting was determined by a spectrophotometry at the wavelength of 600 nm.

Yield was calculated from ratio between the lactic acid produced to sugar used during the fermentation.

### Example 1: The Production of the D-Lactic Acid Using the Process According to the Invention, Wherein the Cultivation and Fermentation Comprising Shaker Mixing

The preparation of Sporolactobacillus laevolacticus bacteria strain, microbial accession number NITE ABP-02334 used in the cultivation step can be done by adding said bacteria in a solid medium with the following composition per liter: about 10 g of sucrose, about 15 g of yeast extract, about 4 g of ammonium chloride (NH₄Cl), about 0.25 g of dipotassium phosphate (K₂HPO₄), about 0.25 g of dihydrogen phosphate (KH₂PO₄), about 5 g of calcium carbonate (CaCO₃), about 5 g of magnesium sulfate (MgSO₄), about 400 mg of manganese sulfate (MnSO₄), about 20 mg of ferrous sulfate, about 20 mg of sodium chloride (NaCl), and about 20 g of agar. The mixer was incubated at about 37° C. for about 24 hours.

The cultivation for producing the seed culture could be done by diluting the bacteria in said solid medium with about 0.5-2% sodium chloride (NaCl) solution to obtain about 12,000 to 16,000 mg dry cell per liter of bacterial cells. Then, adding about 250 μl of the said diluted bacteria into 250 mL flask containing 25 L of sugarcane juice with sugar concentration about 1% by weight, about 0.38 g of yeast extract, about 0.10 g of ammonium chloride, about 6.25 mg of dipotassium phosphate, about 6.25 mg of potassium dihydrogen phosphate, about 0.13 g of calcium carbonate, about 10 mg of magnesium sulfate, about 0.5 mg of manganese sulfate, about 0.5 mg of ferrous sulfate, and about 0.5 mg of salt. According to this step, the starting concentration of bacterial cells about 120-160 mg dry cell per liter could be obtained. Then, the cultivation was performed for obtaining seed culture at the temperature about 37° C. under an aerobic condition. The shaker mixing rate was about 200 rpm for about 5 hours until the final concentration of bacterial cells was about 1,000 to 1,400 mg dry cell per liter.

After that, the said seed culture was fermented by adding about 25 mL of sugarcane juice with the sugar concentration about 20% by weight. The pH was controlled to be about 5.5 to 6.5 with calcium carbonate. The fermentation was done at the temperature about 37° C. under a microaerobic condition. The shaker mixing speed was about 250 rpm for about 48 hours.

### Example 2: The Production of the D-Lactic Acid with the Process According to the Invention, Wherein the Cultivation Step Comprising the Shaker Mixing Step

The seed culture was prepared according to the method as described in example 1. After that, the seed culture was fermented according to the method as described in example 1 without the shaker mixing.

### Example 3: The Production of the D-Lactic Acid with the Process According to the Invention, Wherein the Cultivation and Fermentation Steps Comprising the Shaker Mixing Step, and the Fermentation Operated in 5 L Fermenter

The cultivation for producing the seed culture could be done by diluting the said bacteria in a solid medium obtained from steps as described in example 1 with about 0.5-2% sodium chloride (NaCl) solution to obtain the concentration about 12,000 to 16,000 mg dry cell per liter of bacterial cells. Then, adding about 750 μL of the said diluted bacteria into 500 mL flask containing 75 L of sugarcane juice with the sugar concentration about 1% by weight, about 1.14 g of the yeast extract, about 0.30 g of ammonium chloride, about 18.75 mg of dipotassium phosphate, about 18.75 mg of potassium dihydrogen phosphate, about 0.39 g of calcium carbonate, about 30 mg of magnesium sulfate, about 1.50 mg of manganese sulfate, about 1.50 mg of ferrous sulfate, and about 1.50 mg of salt. According to this step, the starting concentration of bacterial cells about 120-160 mg dry cell per liter could be obtained. Then, the cultivation was performed for obtaining the cell culture at the temperature about 37° C. The pH was controlled by calcium carbonate to be about 5.5 to 6.5 under an aerobic condition. The shaker mixing rate was about 200 rpm until the final concentration of bacterial cells was about 1,000 to 1,400 mg dry cell per liter. After that, the obtained solution was added into 5 L fermenter comprising about 1.5 L of sugarcane juice with the sugar concentration about 1% by weight, about 22.5 g of the yeast extract, about 6.0 g of ammonium chloride, about 0.38 g of dipotassium phosphate, about 0.38 g of potassium dihydrogen phosphate, about 7.5 g of calcium carbonate, about 600 mg of magnesium sulfate, about 30 mg of manganese sulfate, about 30 mg of ferrous sulfate, and about 30 mg of salt. After that, the bacteria were cultivated at the temperature about 37° C., and pH was controlled by calcium carbonate to be about 5.5 to 6.5 under an aerobic condition. Air was added at the rate of 1.5 L/min. The shaker mixing speed was about 300 rpm until the concentration of bacterial cells were about 1,000 to 1,400 mg dry weight per liter.

After that, the seed culture prepared from above steps was fermented by adding about 1.5 L of sugarcane juice with sugar concentration about 20% by weight. The pH was controlled to be about 5.5 to 6.5 with calcium carbonate. The fermentation was done at the temperature about 37° C. under a microaerobic condition. The shaker mixing speed was about 300 rpm for about 45 hours.

Table 1 shows the production of D-lactic acid of the Sporolactobacillus laevolacticus bacterial strain, microbial accession number NITE ABP-02334 by fermentation process with sugarcane juice according to this invention.

| | Maximum | Amount | | | Optical |
|---|---|---|---|---|---|
| | cell | of D- | | Residual | purity of |
| | concentration | lactic acid | Yield | sugar | D-lactic |
| Example | (g/L) | (g/L) | (g/L) | (g/L) | acid |
| Example 1 | 4.4 | 70 | 0.9 | 13.7 | 98% |
| Example 2 | 2.9 | 82 | 0.9 | 14.3 | 99% |
| Example 3 | 2.7 | 117 | 1.0 | 15.95 | 99% |

From table 1, it was found that example 1, 2, and 3 which operated the cultivation of the Sporolactobacillus laevolacticus with sugarcane juice and fermented the obtained seed culture with the method according to this invention gave good D-lactic acid. In other word, it provided the maximum amount of the D-lactic acid about 117 g/L, the maximum yield per starting sugar about 1.0 g/g, and the optical purity of the obtained D-lactic acid was about 99%.

### BEST MODE OF THE INVENTION

Best mode of the invention is as provided in the description of the invention.

1: A fermentation process for producing D-lactic acid or a salt thereof, the fermentation process comprising:
(a) cultivating Sporolactobacillus laevolacticus bacteria strain, microbial accession number NITE ABP-02334, in sugarcane juice, to obtain a seed culture; and
(b) fermenting the seed culture in sugarcane juice;
wherein (a) is operated for a time such that a final concentration of the Sporolactobacillus laevolacticus bacteria strain in (a) is in a range of from 400 to 1,600 mg of dry cell per liter.

2: The fermentation process of claim 1, wherein the final concentration of the Sporolactobacillus laevolacticus bacteria strain in (a) is in a range of from 1,000 to 1,400 mg of dry cell per liter.

3: The fermentation process of claim 1, wherein (a) is operated under an aerobic condition.

4: The fermentation process of claim 1, wherein a starting concentration of the Sporolactobacillus laevolacticus bacteria strain in (a) is in a range of from 40 to 240 mg of dry cell per liter.

5: The fermentation process of claim 4, wherein the starting concentration is in a range of from 120 to 160 mg of dry cell per liter.

6: The fermentation process of claim 1, wherein (b) is operated under a mircoaerobic condition.

7: The fermentation process of claim 1, wherein (b) is operated for a time in a range of from 12 to 50 hours.

8: The fermentation process of claim 7, wherein (b) is operated for a time in a range of from 45 to 50 hours.

9: The fermentation process of claim 1, wherein the sugarcane juice in (b) comprises a sugar and a sugar concentration in (b) is in a range of from 4 to 15% by volume.

10: The fermentation process of claim 1, wherein (a) and (b) are operated at a temperature in a range of from 35 to 40° C.

11: The fermentation process of claim 10, wherein (a) and (b) are operated at a temperature of 37° C.

12: The fermentation process of claim 1, wherein the D-lactic acid or the salt thereof has an optical purity of more than 95%.

13: The fermentation process of claim 12, wherein the optical purity is more than 99%.
